(19) United States
(12) Patent Application Publication
(10) Pub. No.: US 2005/0021065 A1
(43) Pub. Date: Jan. 27, 2005

(54) Ultrasonic treatment apparatus

(75) Inventors: Norihiro Yamada, Tokyo (JP); Yoshitaka Honda, Tokyo (JP); Hitoshi Karasawa, Tokyo (JP); Ken Fujinuma, Fujimi-shi (JP); Mitsumasa Okada, Tokyo (JP); Takeaki Nakamura, Tokyo (JP)

Correspondence Address: SCULLY SCOTT MURPHY & PRESSER, PC, 400 GARDEN CITY PLAZA, GARDEN CITY, NY, 11530, US

(73) Assignee: OLYMPUS CORPORATION, TOKYO (JP)

(21) Appl. No.: 10/896,352

(22) Filed: Jul. 21, 2004

(30) Foreign Application Priority Data
Jul. 24, 2003 (JP) 2003-201236

(51) Int. Cl. A61B 17/32

(52) U.S. Cl. 606/169

(57) ABSTRACT

An ultrasonic treatment apparatus comprises an ultrasonic transmitting member which has a treatment portion for treating a target portion and which transmits ultrasonic vibrations to the treatment portion, a transducer which is connected to the ultrasonic transmitting member and includes a first element for vibrating the ultrasonic transmitting member in an axial direction thereof and a second element for vibrating the ultrasonic transmitting member in a torsional direction thereof, a rotation driving portion which freely rotates the transducer, a first driving portion which drives the first element in the transducer, a second driving portion which drives the second element in the transducer, and a control portion which independently controls the first driving portion, the second driving portion, and the rotation driving portion.

This application claims benefit of Japanese Application No. 2003-201236 filed in Japan on Jul. 24, 2003, the contents of which are incorporated by this reference.

### BACKGROUND OF THE INVENTION

1. Field of the Invention

The present invention relates to an ultrasonic treatment apparatus which can destroy the tissue such as the calculus or bone by using a transducer.

2. Description of the Related Art

Recently, various operation apparatuses for endoscope curing of the calculus in the urinary tract and the like are developed. In the operation apparatuses, an ultrasonic treatment apparatus (or ultrasonic lithotripsy apparatus) is widely used. The ultrasonic treatment apparatus transmits ultrasonic vibrations to a probe (ultrasonic transmitting member) and finely destroys the calculus at the probe edge thereof. The ultrasonic treatment apparatus has a feature that it does not influence on its peripheral tissue. The soft tissue absorbs the vibrations and is not influenced from the vibrations. However, the hard tissue such as the calculus or bone remarkably receives the vibration energy.

For example, Japanese Examined Patent Application Publication No. 06-087856 discloses one of the above-mentioned conventional ultrasonic treatment apparatuses, in which a cover is provided around a probe for transmitting the ultrasonic vibrations so as to protect an endoscope channel and the probe edge is exposed from the cover edge to destroy the calculus.

Meanwhile, Japanese Unexamined Patent Application Publication No. 2002-209906 discloses another conventional ultrasonic treatment apparatus, in which the vibrations for rotation in the axial direction, namely, torsional vibrations are generated so as to destroy the tissue such as the calculus or bone.

Further, U.S. Pat. No. 5,116,343 discloses another conventional ultrasonic treatment apparatus, in which the lateral vibrations and the vibrations for rotation in the axial direction, that is, the torsional vibrations are generated so as to destroy the tissue such as the calculus or bone.

### SUMMARY OF THE INVENTION

According to the present invention, an ultrasonic treatment apparatus comprises an ultrasonic transmitting. member which has a treatment portion for treating a target portion and transmits ultrasonic vibrations to the treatment portion, a transducer which is connected to the ultrasonic transmitting member and includes a first element for vibrating the ultrasonic transmitting member in an axial direction thereof and a second element for vibrating the ultrasonic transmitting member in a torsional direction thereof, a rotation driving portion which freely rotates the transducer, a first driving portion which drives the first element in the transducer, a second driving portion which drives the second element in the transducer, and a control portion which independently controls the first driving portion, the second driving portion, and the rotation driving portion.

Further, according to the present invention, an ultrasonic treatment apparatus comprises a transducer which generates the ultrasonic vibrations, and a treatment portion for treating a target portion. The treatment portion is connected to the transducer so that the ultrasonic vibrations generated by the transducer are transmitted, and at least a part of the treatment portion being provided with non-circular-shaped cross section in the direction perpendicular to the longitudinal direction thereof.

Furthermore, according to the present invention, an ultrasonic treatment apparatus comprises an ultrasonic transmitting member which has a treatment portion for treating the tissue at one end thereof, and which transmits ultrasonic vibrations to the treatment portion, a transducer which is connected to the ultrasonic transmitting member and includes a first piezoelectric element for vibrating the ultrasonic transmitting member in an axial direction of the ultrasonic transmitting member and a second piezoelectric element for vibrating the ultrasonic transmitting member in a torsional direction of the ultrasonic transmitting member, the first piezoelectric element and the second piezoelectric element being laminated in an axial direction of the ultrasonic transmitting member, an electromagnetic motor which freely rotates the entire transducer, a first driving portion which drives the first element, a second driving portion which drives the second element, and a control portion which independently controls the power which is supplied to the first piezoelectric element, the second piezoelectric element, and the electromagnetic motor.

### BRIEF DESCRIPTION OF THE DRAWINGS

FIG. 1 is a diagram showing the entire structure of an ultrasonic treatment apparatus according to the first embodiment of the present invention;

FIG. 2 is a diagram showing the entire structure of an ultrasonic treatment apparatus upon detaching a motor portion shown in FIG. 1;

FIG. 3 is an enlarged view showing the structure of a treatment portion shown in FIG. 1;

FIG. 4 is an explanatory diagram showing the periphery of a connecting portion between an ultrasonic transmitting member and a horn shown in FIG. 1;

FIG. 5 is a circuit block diagram showing the structure of a transducer, a motor portion, a portion for generating the longitudinal vibrations and torsional vibrations in the transducer, and a portion for freely rotating the transducer;

FIG. 6 is a front view showing an operating panel of a signal generating device shown in FIG. 1;

FIG. 7 is an explanatory diagram showing the operation of a treatment portion using the longitudinal vibrations and the motor rotation;

FIG. 8 is an explanatory diagram showing the operation of the treatment portion using the torsional vibrations and the motor rotation;

FIG. 9 is an enlarged view showing the structure of a treatment portion in an ultrasonic treatment apparatus according to the second embodiment of the present invention;

FIG. 10 is an enlarged view showing a modification of the treatment portion shown in FIG. 9;

FIG. 11 is an enlarged view showing the structure of a treatment portion in an ultrasonic treatment apparatus when an advance and return portion returns according to the third embodiment;

FIG. 12 is an enlarged view showing the treatment portion when the advance and return portion advances to the edge side shown in FIG. 11;

FIG. 13 is an enlarged view showing a modification of the treatment portion shown in FIG. 11 when the advance and return portion returns;

FIG. 14 is an enlarged view showing the treatment portion when the advance and return portion advances to the edge side shown in FIG. 13; and

FIG. 15 is an enlarged view showing a modification of the treatment portion shown in FIG. 13.

### DETAILED DESCRIPTION OF THE PREFERRED EMBODIMENTS

Hereinbelow, a description is given of preferred embodiments of the present invention with reference to the drawings.

First Embodiment

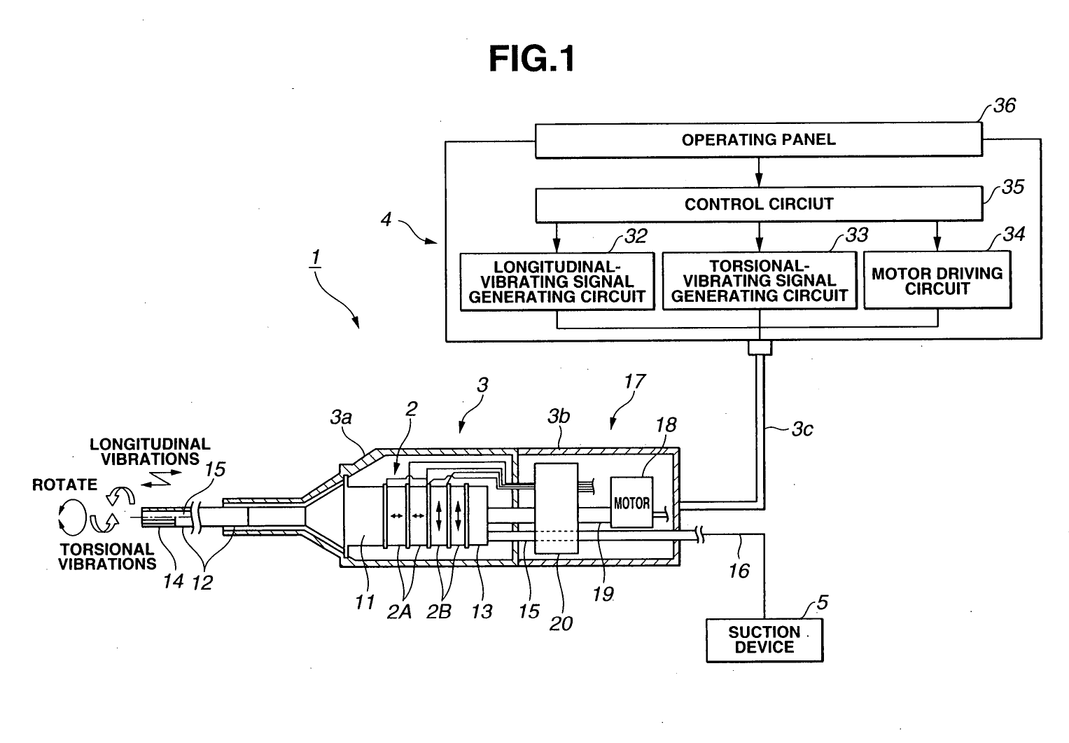

FIGS. 1 to 8 show an ultrasonic treatment apparatus according to the first embodiment of the present invention.

Referring to FIG. 1, an ultrasonic treatment apparatus 1 according to the first embodiment of the present invention comprises: an ultrasonic hand piece 3 including a transducer 2 for generating vibrations; an ultrasonic driving signal generating device (referred to as a signal generating device) 4 which applies a driving signal for generating the ultrasonic vibrations in the ultrasonic hand piece 3; and a suction device 5 which sucks the tissue via a suction channel formed to the ultrasonic hand piece 3, which will be described later.

The ultrasonic hand piece 3 includes, in a casing 3a for vibrator on the rear end side, the transducer 2 which can freely be rotated. Further, the ultrasonic hand piece 3 includes: a horn 11 which amplifies the ultrasonic vibrations generated by the transducer 2; and a long ultrasonic transmitting member 12 which is tightened to the transducer 2 and transmits the ultrasonic vibrations via the horn 11. Reference numeral 13 denotes a lined plate. The lined plate 13 and the horn 11 sandwich a first piezoelectric element (a first element) 2A which vibrates the ultrasonic transmitting member 12 in its axial direction (hereinafter, referred to as longitudinal-vibrations) and a second piezoelectric element (a second element) 2B which vibrates the ultrasonic transmitting member in its torsional direction (hereinafter, referred to as torsional vibrations), both of which will be described later, thereby constituting the transducer 2.

The ultrasonic transmitting member 12 has, at the tip thereof, a treatment portion 14 for treating a target portion (destroying the tissue such as the calculus or bone) by the ultrasonic vibrations generated by the transducer 2. The ultrasonic transmitting member 12 further has a suction channel 15 which is opened to the treatment portion 14 and sucks the tissue. The suction channel 15 is continuously connected to a suction cable 16 via the horn 11, the transducer 2, and the lined plate 13. The suction cable 16 is extended from the rear end portion of the ultrasonic hand piece 3. The suction cable 16 is detachably connected to the suction device 5. The suction cable 16 sucks the tissue which is sucked from the treatment portion 14 in the ultrasonic transmitting member 12.

The ultrasonic hand piece 3 has a motor portion 17 on the back surface side of the transducer 2. The motor portion 17 freely rotates the transducer 2 together with the ultrasonic transmitting member 12.

The motor portion 17 is accommodated in a motor casing 3b.

The motor portion 17 comprises: a rotatable electromagnetic motor (hereinafter, referred to as a motor) 18; a rotating shaft 19; and a slip ring 20.

The rotating shaft 19 transmits the rotation of the motor 18 by the connection to the lined plate 13 in the transducer 2. The slip ring 20 prevents the twisting of the suction channel 15 and a signal line connected to the transducer 2, upon rotating the motor 18.

In the ultrasonic hand piece 3, a driving cable 3c is detachably connected to the signal generating device 4. In the driving cable 3c, a signal line connected to the transducer 2 and a signal line connected to the motor portion 17 are inserted.

Further, in the ultrasonic hand piece 3, a driving signal is applied to the motor 18 of the motor portion 17 by a driving signal from the signal generating device 4, and the transducer 2 is freely rotated together with the ultrasonic transmitting member 12. Simultaneously, in the ultrasonic hand piece 3, a driving signal for ultrasonic vibrations from the signal generating device 4 is applied to the transducer 2. Then, in the transducer 2, the longitudinal vibrations, torsional vibrations, or the combining vibrations thereof are generated. The vibration energy is transmitted to the treatment portion 14 via the ultrasonic transmitting member 12. When the treatment portion 14 comes into contact with the hard tissue such as the calculus or bone, the ultrasonic vibration energy is applied to the tissue and the tissue is broken.

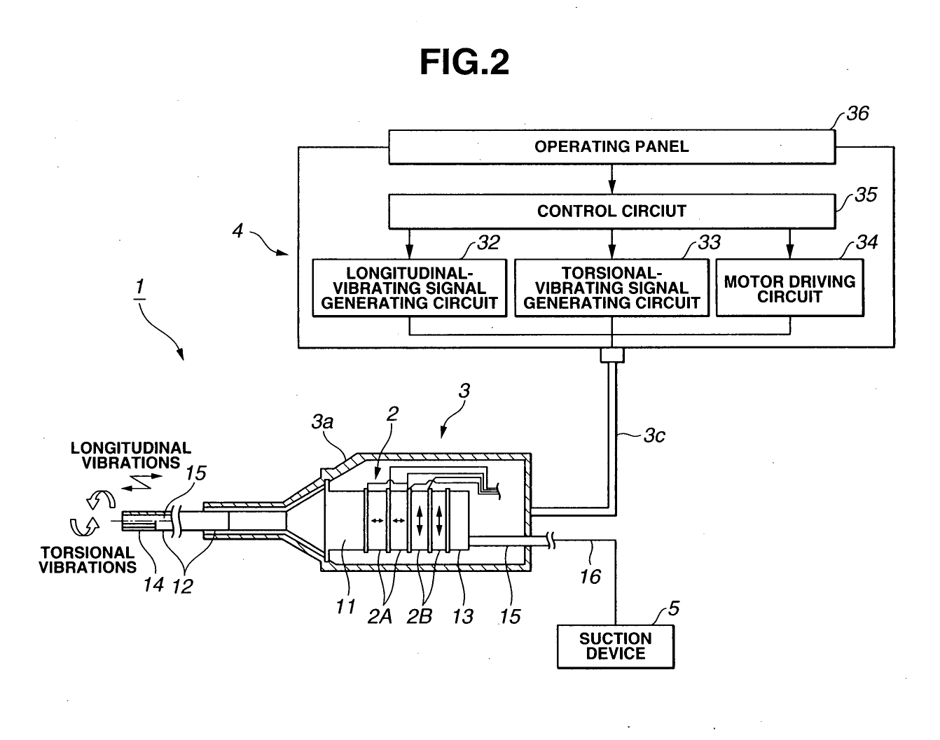

When the treatment target is only the relatively soft tissue such as the muscle tissue, internal organ, or cartilage, the ultrasonic hand piece 3 can perform the treatment only by the ultrasonic vibrations. In this case, referring to FIG. 2, the motor portion 17 is detached from the ultrasonic hand piece 3, and the transducer 2 is directly connected to the signal generating device 4.

Here, according to the first embodiment, the torsional vibrations are actively applied as well as the longitudinal vibrations. Thus, the hard tissue can effectively be broken.

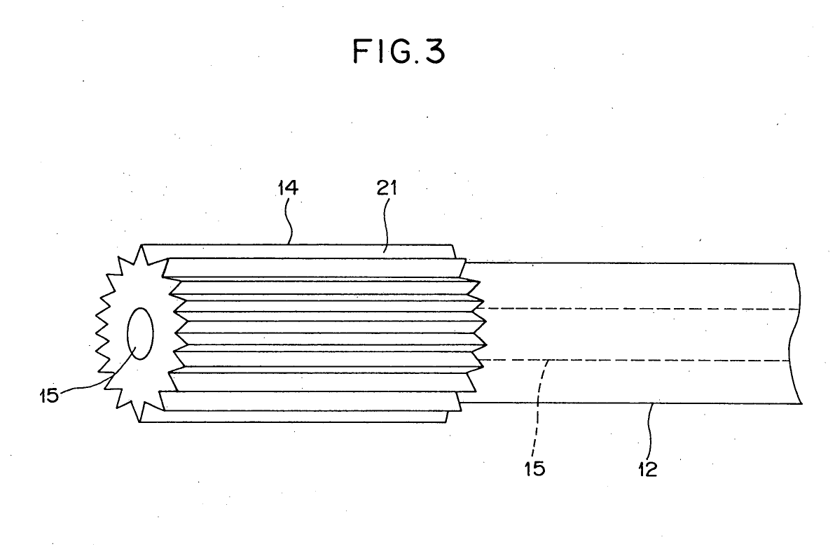

FIG. 3 is an enlarged view showing the structure of the treatment portion 14 shown in FIG. 1.

Referring to FIG. 3, the treatment portion 14 has a groove 21 on the outer periphery. The groove 21 can destroy the tissue by the edge thereof.

Then, the treatment portion 14 uses the torsional vibrations, thereby applying the vibration energy to the calculus without moving the calculus to another place.

The suction channel 15 is opened to the treatment portion 14. The treatment portion 14 sucks the tissue from the opening of the suction channel 15. The tissue through the suction channel 15 is discharged to the suction device 5 outside of the hand piece.

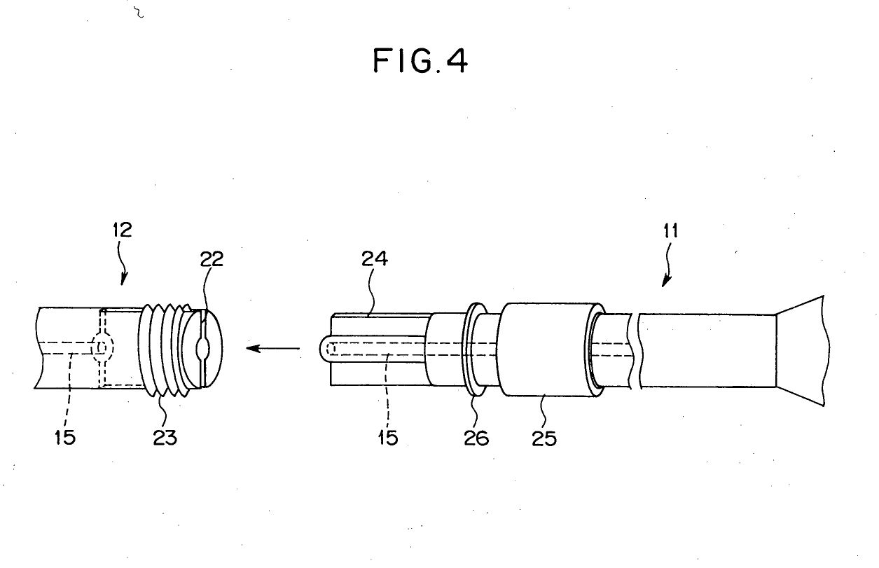

FIG. 4 is an explanatory diagram showing the periphery of a connecting portion between the ultrasonic transmitting member 12 and the horn 11 shown in FIG. 1.

Referring to FIG. 4, a cave portion 22 is formed on the base end side of the ultrasonic transmitting member 12. A male screw portion 23 is formed on the base end side of the ultrasonic transmitting member 12. A projected portion 24 fit into the cave portion 22 in the ultrasonic transmitting member 12 is formed on the edge side of the horn 11. A ring member 25 is arranged on the edge side of the horn 11. In the ring member 25, a female screw portion (not shown) screwed to the male screw portion 23 in the ultrasonic transmitting member 12 is formed onto the inner periphery.

The ring member 25 is attached to be moved in the axial direction on the edge side of the horn 11. The position of the ring member 25 is regulated by a stopper member 26. Incidentally, the suction channel 15 is arranged in the center of the cave portion 22 and the projected portion 24.

The projected portion 24 of the horn 11 is fit into the cave portion 22 of the ultrasonic transmitting member 12. Further, the ring member 25 is screwed to the male screw portion 23 of the ultrasonic transmitting member 12.

Thus, the ultrasonic hand piece 3 regulates the rotation in the axial direction of the horn 11 and the ultrasonic transmitting member 12 jointed thereto.

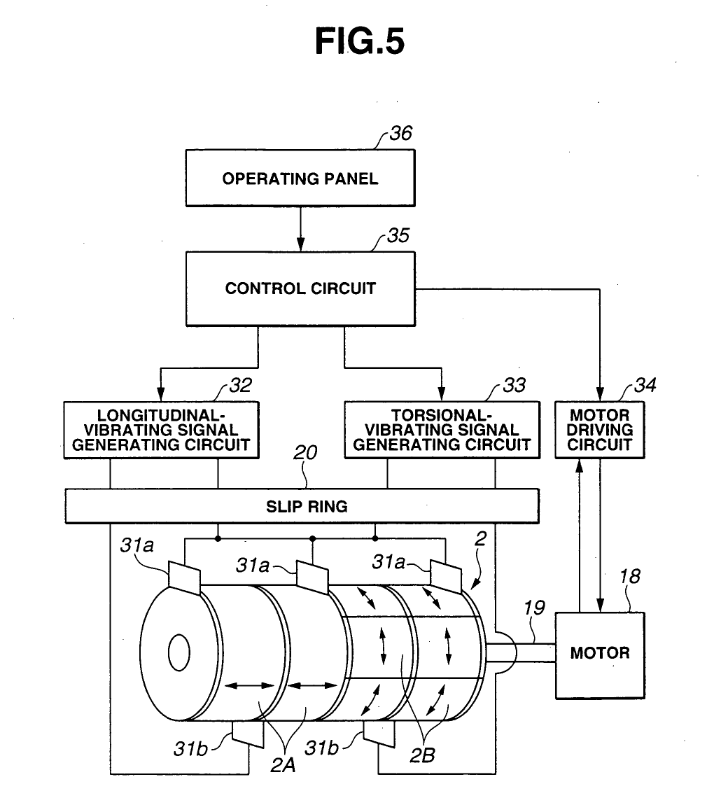

FIG. 5 is a circuit block diagram showing the structure of the transducer 2, the motor portion 17, a portion for generating the longitudinal vibrations and the torsional vibrations in the transducer 2, and a portion for freely rotating the transducer 2.

According to the first embodiment, the transducer 2 is formed by laminating a plurality of piezoelectric elements. Here, a description is given of the case of the transducer 2 comprising four piezoelectric elements.

Two of the four piezoelectric elements are, as the first element, the longitudinal-vibration piezoelectric elements 2A which are polarized to generate the strain in the longitudinal direction (in the axial direction of the ultrasonic transmitting member 12). Other piezoelectric elements are, as the second element, the torsional vibration piezoelectric elements 2B which are polarized to generate the strain in the torsional direction (in the torsional direction of the ultrasonic transmitting member 12).

Electrodes 31a and 31b are arranged onto the both surfaces of the four piezoelectric elements. A part of the electrodes 31a and 31b are projected to the outside on both the surfaces of the piezoelectric elements so as to easily connect the signal line to which the driving signal is applied.

Meanwhile, the signal generating device 4 comprises: a longitudinal-vibrating signal generating circuit 32 which generates a driving signal for the longitudinal vibrations as a first driving portion; and a torsional-vibrating signal generating circuit 33 which generates a driving signal for torsional vibrations as a second driving portion.

Further, the signal generating device 4 comprises a motor driving circuit 34. The motor driving circuit 34 generates a driving signal of the motor 18 in the motor portion 17. The motor portion 17 and the motor driving circuit 34 form a rotation driving portion.

Furthermore, the signal generating device 4 comprises a control circuit (control portion) 35. The control circuit 35 independently controls the longitudinal-vibrating signal generating circuit 32, the torsional-vibrating signal generating circuit 33, and the motor driving circuit 34.

The control circuit 35 selects a vibration mode which is generated by the operation of an operating panel 36. That is, the control circuit 35 arbitrarily controls the on/off operation and the intensity of a longitudinal-vibration signal, a torsional-vibration signal, and a motor signal according to the selection by the operating panel 36.

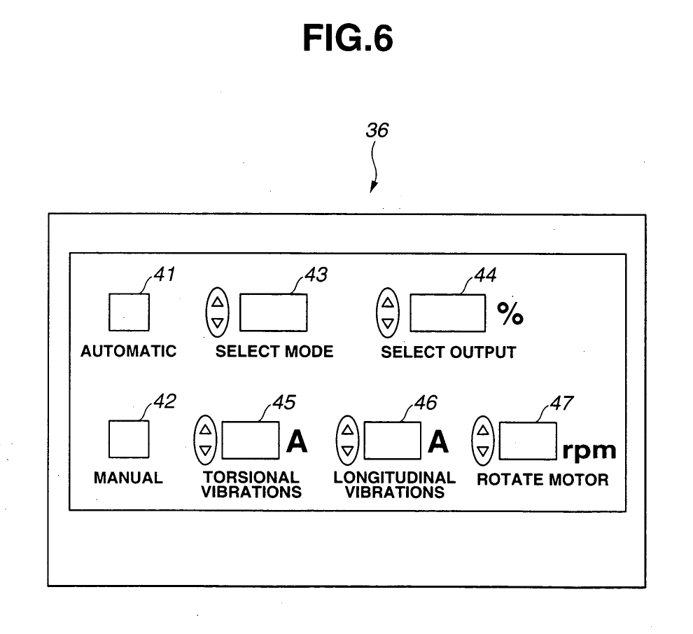

Referring to FIG. 6, the operating panel 36 comprises setting buttons (or setting portions) in vibration modes.

Further, referring to FIG. 6, the operating panel 36 comprises: an automatic button 41; a manual button 42; a mode selecting button 43; an output setting button 44; a torsional-vibration output adjusting button 45; a longitudinal-vibration output adjusting button 46; and a motor rotating speed adjusting button 47.

Here, the signal generating device 4 selects the desired mode from modes 1 to 6 which are preset as shown in Table 1 by pressing the automatic button 41.

TABLE 1

| | | | Motor | |
|---|---|---|---|---|
| | Longitudinal | Torsional | rotation | |
| Mode | Vibration (A) | vibration (A) | (rpm) | Application |
| 1 | 1.0 | 0 | 0 | Perforation, |
| | | | | emulsification |
| | | | | and aspiration |
| | | | | (soft tissue) |
| 2 | 1.0 | 0 | 1000 | Perforation |
| | | | | (hard tissue) |
| 3 | 0 | 1.0 | 0 | Cutting |
| | | | | (soft tissue) |
| 4 | 0 | 1.0 | 1000 | Cutting |
| | | | | (hard tissue) |
| 5 | 0.5 | 0.5 | 0 | Perforation and |
| | | | | cutting |
| | | | | (soft tissue) |
| 6 | 0.5 | 0.5 | 1000 | Perforation and |
| | | | | cutting |
| | | | | (hard tissue) |

Values in modes described in Table 1 indicate current values supplied to the longitudinal-vibration piezoelectric element 2A and the torsional vibration piezoelectric element motor the output of 100% and the number of rotations of the motor 18. Although applications shown in Table 1 indicate tentatives for selecting the modes, they are examples and the modes may arbitrarily be selected depending on the situation of the treatment target portion.

The signal generating device 4 selects one of the modes 1 to 6 by the mode selecting button 43 and then can set the output at the interval of 10 to 100% by the output setting button 44.

Meanwhile, in the signal generating device 4, the manual button 42 is pressed and then the current values supplied to the longitudinal-vibration piezoelectric element 2A and the torsional vibration piezoelectric element 2B and the number of rotations of the motor 18 can individually be set by the longitudinal-vibration output adjusting button 46, the torsional-vibration output adjusting button 45, and a motor rotating number adjusting button 47.

A setting range of the longitudinal-vibration output adjusting button 46 and the torsional-vibration output adjusting button 45 is 0 to 1.0 A. A setting range of the motor rotating number adjusting button 47 is 0 to 1,000 rpm. The adjusting buttons 22 to 24 enters a state of the ultrasonic vibrations or an off operation of the motor by selecting the current value 0 A or 0 rpm.

A description is given of the operation with the above-mentioned structure according to the first embodiment of the present invention.

First, the ultrasonic treatment apparatus 1 which connects the motor portion 17 shown in FIG. 1 is used and the hard tissue such as the calculus or bone is treated.

An operator confirms the treatment target tissue in the patient by a hard endoscope (not shown). Further, the operator inserts the ultrasonic transmitting member 12 in the ultrasonic treatment apparatus 1 shown in FIG. 1 via a channel for inserting the treatment tool arranged in the hard endoscope or a trocar.

Furthermore, the operator presses the treatment portion 14 in the ultrasonic transmitting member 12 to the tissue as the treatment target tissue. Then, the operator presses the automatic button 41 in the operating panel 36 described with reference to FIG. 6. The operator further selects a mode 2 in Table 1 by using the mode selecting button 43. Here, the mode 2 (the Perforation mode) indicates the longitudinal vibrations and the motor rotation.

Then, the control circuit 35 controls the longitudinal-vibrating signal generating circuit 32 and the motor driving circuit 34. The longitudinal-vibrating signal generating circuit 32 generates a driving signal for longitudinal vibrations and outputs the generated signal to the transducer 2. Simultaneously, the motor driving circuit 34 generates a motor driving signal and outputs the generated signal to the motor 18.

Then, the transducer 2 is vibrated by the vibrations of the longitudinal-vibration piezoelectric element 2A to which the driving signal for the longitudinal vibrations is applied, and is rotated by rotating force of the motor 18 transmitted through the rotating shaft 19. Further, the longitudinal vibrations generated by the transducer 2 are transmitted to the treatment portion 14 in the ultrasonic transmitting member 12.

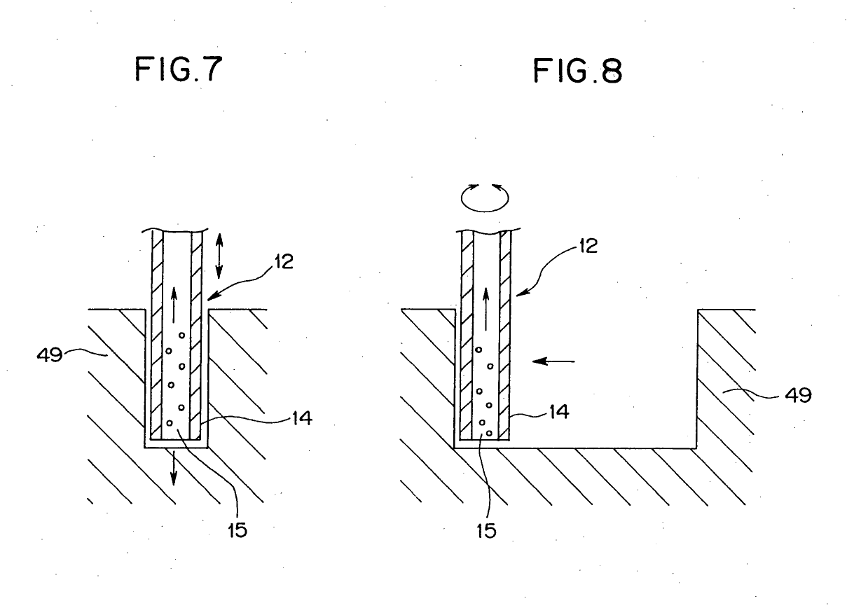

Referring to FIG. 7, in the treatment portion 14, the ultrasonic transmitting member 12 is longitudinally vibrated in the axial direction, thereby iteratively impacting the edge of the treatment portion 14 to a tissue 49 as the treatment target tissue. In addition, the groove 21 in the treatment portion 14 cuts the tissue 49 of the treatment target tissue, thereby enable the perforation. Cutting waste is discharged from the suction channel 15 to the suction device 5.

Meanwhile, the operator selects the mode 4 in Table 1 by using the mode selecting button 43. Here, the mode 4 (the second cutting mode) corresponds to the combination of the torsional vibrations and the motor rotation.

Then, the control circuit 35 controls the torsional-vibrating signal generating circuit 33 and the motor driving circuit 34. The torsional-vibrating signal generating circuit 33 generates the driving signal for torsional vibrations and outputs the generated signal to the transducer 2. Simultaneously, the motor driving circuit 34 generates the motor driving signal and outputs the generated signal to the motor 18.

Then, in the transducer 2, the driving signal for torsional vibrations is applied to the torsional vibration piezoelectric element 2B, thereby torsionally vibrating the transducer 2. Further, the transducer 2 is rotated by rotating force of the motor 18 transmitted through the rotating shaft 19. Simultaneously, the torsional vibrations generated by the transducer 2 are transmitted to the treatment portion 14 in the ultrasonic transmitting member 12.

Referring to FIG. 8, in the treatment portion 14, the ultrasonic transmitting member 12 reciprocates in the diameter several tens μm onto the tissue 49 as the treatment target tissue by the torsional vibrations. In addition, the groove 21 of the treatment portion 14 cuts the tissue 49 by the rotation of the motor 18, thereby smoothly cutting the hard tissue.

In the mode 4, the output is set to 100%, the vibration speed of the torsional vibrations is approximately 5 m/sec, and the motor rotating speed is approximately 0.2 m/sec.

As mentioned above, since the rotating speed of the motor 18 is slower than the torsional-vibration speed, the ultrasonic hand piece 3 prevents the tissue 49 as the treatment target tissue from being jerked caused by the treatment portion 14 during the treatment. In the ultrasonic hand piece 3, the tissue 49 as the treatment target tissue is always in contact with the treatment portion 14, thereby performing-the treatment of the tissue more easily.

Further, in the case of the iterate treatment of the perforation and cutting of the hard tissue or simultaneously performing them, the mode 6 in Table 1 is effective.

The mode 6 (the second perforation and cutting mode) is selected by the mode selecting button 43. Thus, the ultrasonic hand piece 3 enables the perforation by the longitudinal vibrations and the motor rotation and the cutting by the torsional vibrations and the motor rotation.

When the tissue 49 as the treatment target tissue is the soft tissue such as the skin, mucous membrane, muscle, organ, or cartilage, the rotation of the motor 18 is not necessary because the load to the treatment portion 14 is low during the treatment.

Then, in the case of the perforation, the mode 1 (the perforation, emulsification and aspiration mode) may be selected. In the case of cutting, the mode 3 (the first cutting mode) may be selected. In the case of the perforation and cutting, the mode 5 (the first perforation and cutting mode) may be selected. These selections may use the mode selecting button 43.

If the treatment target is the bone or calculus, the treatment time is longer as compared with the ON operation of the motor rotation depending on the size or shape. However, in the case of the modes 1, 3, and 5 in the OFF operation of the motor rotation, the treatment is possible. In the case of the extremely soft tissue such as the muscle or organ, only the mode 1 enables the perforation and the incision.

Referring to FIG. 2, in the case of the ultrasonic hand piece 3 from which the motor portion 17 is detached, the modes 2, 4, and 6 in the on operation of the motor rotation are not selected.

As a result, the ultrasonic treatment apparatus 1 according to the first embodiment can perform the various treatments of the tissue by freely operating the output of the longitudinal vibrations, torsional vibrations and motor rotation. Further, in the ultrasonic treatment apparatus 1 according to the first embodiment, the motor rotating speed is lower than the vibration speed of the torsional vibrations. Thus, it is possible to prevent the movement of the treatment target tissue by the treatment portion 14 during the treatment, and to provide the constant contact of the treatment portion 14 to the treatment target tissue.

Therefore, the ultrasonic treatment apparatus 1 according to the first embodiment can arbitrarily change the amplitudes of the longitudinal vibrations and the amplitudes of the torsional vibrations depending on the treatment tissue.

Second Embodiment

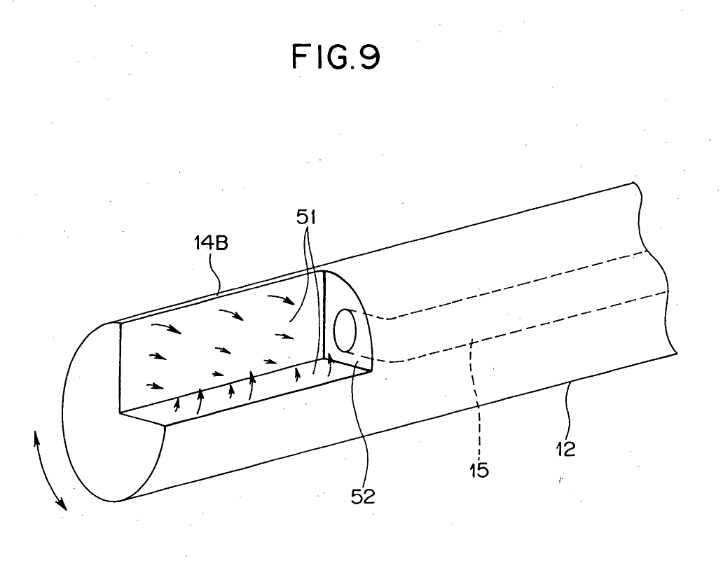

FIGS. 9 and 10 show an ultrasonic treatment apparatus according to the second embodiment of the present invention.

According to the second embodiment, the cavitation generating surface for generating the cavitation, which is caused by the torsional vibrations, is formed to the treatment portion 14. Other structures are the same as those according to the first embodiment, a description thereof is omitted, and the same components are designated by the same reference numerals.

Referring to FIG. 9, the ultrasonic treatment apparatus according to the second embodiment comprises a treatment portion 14B. The cavitation generating surface is provided at the treatment portion 14B. The cavitation generating surface generates the cavitation due to the torsional vibrations.

The treatment portion 14B has a notch surface 51 that is formed horizontally to its axial direction on the tip side, as the cavitation generating surface. The treatment portion 14B has an opening surface 52 having the opening of the suction channel 15 on the base end side of the notch surface 51.

The treatment portion may be structured as shown in FIG. 10.

That is, a treatment portion 14C has a notch surface 51c, which is provided with semi-circular-shaped cross section in a direction perpendicular to the axial direction of the treatment portion 14, on the tip side thereof as the cavitation generating surface. The treatment portion 14C has an opening surface 52c having the opening of the suction channel 15 on the base end side of a notch surface 51c.

The treatment portions 14B and 14C can destroy and emulsify the tissue by the cavitation generated at the notch surfaces 51 and 51c.

Other structures are the same as those according to the first embodiment and a description thereof is omitted.

A description is given of the operation with the above-mentioned structure according to the second embodiment.

Similarly to the first embodiment, a description is given of the case of cutting the tissue as the treatment target tissue in the modes 3 to 6 using the torsional vibrations with the ultrasonic treatment apparatus.

Referring to the Table 1, upon cutting the tissue 49 in the modes 3 to 6 with the torsional vibrations, in the treatment portions 14B and 14C, the notch surfaces 51 and 51c are horizontal to the axial direction of the treatment portion 14 and therefore the cavitation is efficiently emitted due to the torsional vibrations from the notch surfaces 51 and 51c.

As a result, the treatment portions 14B and 14C can fast perform the treatment by destroying and emulsifying the tissue 49 using the cavitation generated from the notch surfaces 51 and 51c as well as by cutting the tissue 49 as the treatment target tissue. Other operations are the same as those according to the first embodiment and therefore a description thereof is omitted.

Thus, the ultrasonic treatment apparatus according to the second embodiment obtains the same advantages as those according to the first embodiment. Further, the tissue can be emulsified and destroyed by using the cavitation using the torsional vibrations.

Third Embodiment

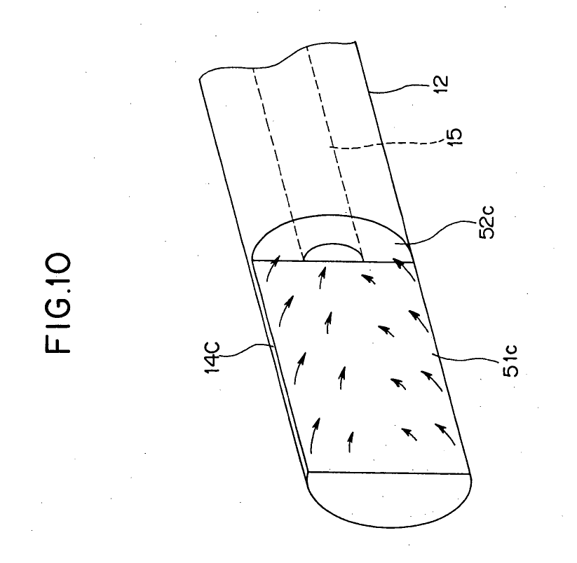

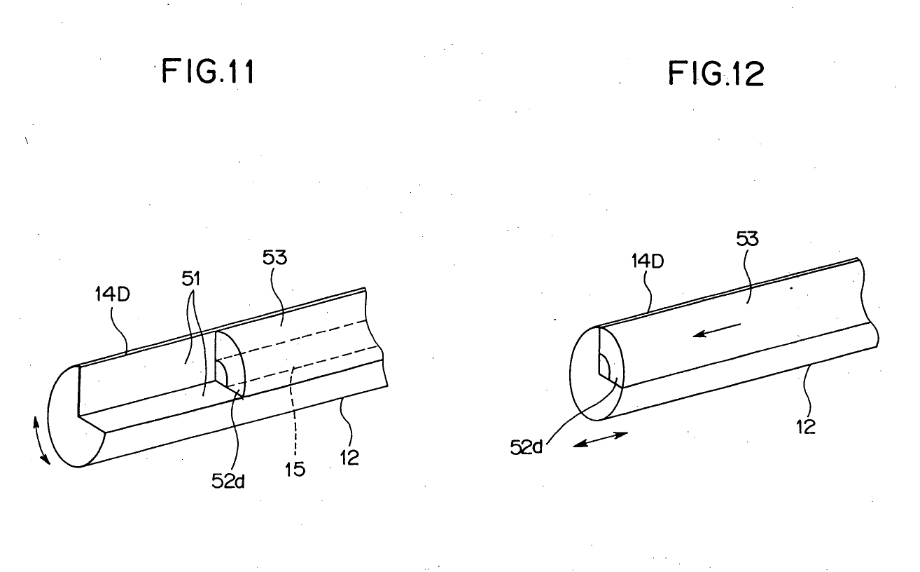

FIGS. 11 to 15 show an ultrasonic treatment apparatus according to the third embodiment of the present invention.

According to the third embodiment, the opening surface according to the second embodiment is slidably provided to the notch surface. Other structures are the same as those according to the second embodiment, therefore, a description thereof is omitted, and the same components are designated by the same reference numerals.

Referring to FIG. 11, the ultrasonic treatment apparatus according to the third embodiment comprises a treatment portion 14D having an advance and return portion (slide portion) 53 on the notch surface 51, which is provided slidably onto the notch surface 51. The advance and return portion 53 has an opening surface 52d having the opening of the suction channel 15 on the tip surface thereof.

The advance and return portion 53 is slidable to the notch surface 51 in the longitudinal direction by driving a linear motor (not shown). In this case, the linear motor is driving controlled under the control of the control circuit 35.

When the treatment portion 14D uses the torsional vibrations in the modes 3 to 6 shown in Table 1 or the torsional vibrations are outputted in the manual mode, the advance and return portion 53 is moved back and the notch surface 51 is exposed.

Meanwhile, in the mode 1 shown in Table 1 or in the case of outputting only the longitudinal vibrations in the manual mode, the linear motor is driving controlled under the control of the control circuit 35 and thus the advance and return portion 53 advances. Then, referring to FIG. 12, the notch surface 51 is hidden.

Other structures are the same as those according to the second embodiment and therefore a description thereof is omitted.

A description is given of the operation with the above-mentioned structure according to the third embodiment.

A description is given of the case of cutting the tissue as the treatment target tissue in the modes 3 to 6 using the torsional vibrations with the ultrasonic treatment apparatus, similarly to the first embodiment.

In the case of cutting the tissue 49 in the modes 3 to 6 using the torsional vibrations as shown in the Table 1, in the treatment portion 14D, the notch surface 51 is horizontal to the axial direction and therefore the cavitation is efficiently emitted due to the torsional vibrations from the notch surface 51.

Therefore, the treatment portion 14D is able to provide a prompt treatment by destroying and emulsifying the tissue 49 using the cavitation generated from the notch surface 51 as well as by cutting the tissue 49 as the treatment target tissue.

Meanwhile, referring to FIG. 7, in the case of perforating the tissue 49 in the mode 1 using only the longitudinal vibrations, the advance and return portion 53 advances in the treatment portion 14D as shown in FIG. 12. The cavitation is uniformly emitted due to the longitudinal vibrations from the tip and the treatment portion 14D perforates the tissue 49. Other structures are the same as those according to the first embodiment and therefore a description is omitted.

The treatment portion 14D has an outer peripheral portion (not shown) including the advance and return portion 53 which has the groove 21 described with reference to FIG. 3 or is drill-shaped. Thus, the hard tissue can effectively be perforated in the mode 2 using the longitudinal vibrations and the motor rotation.

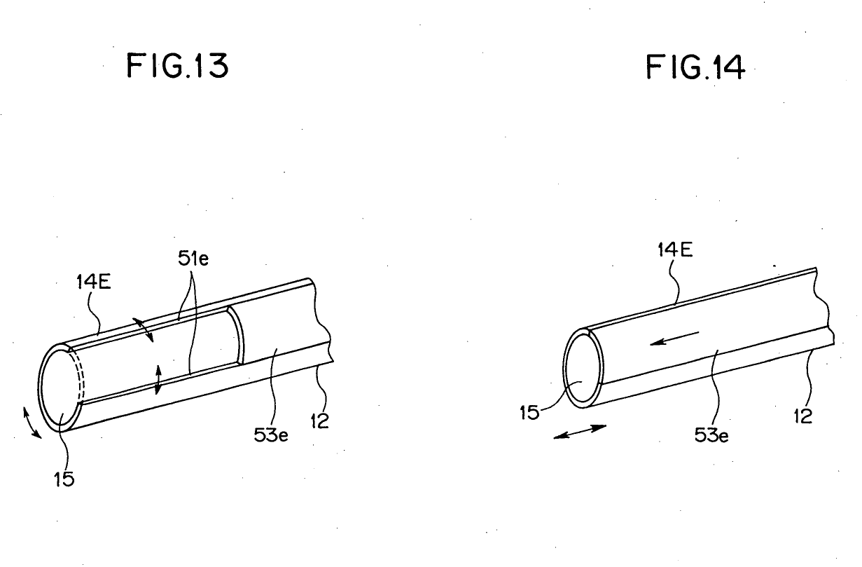

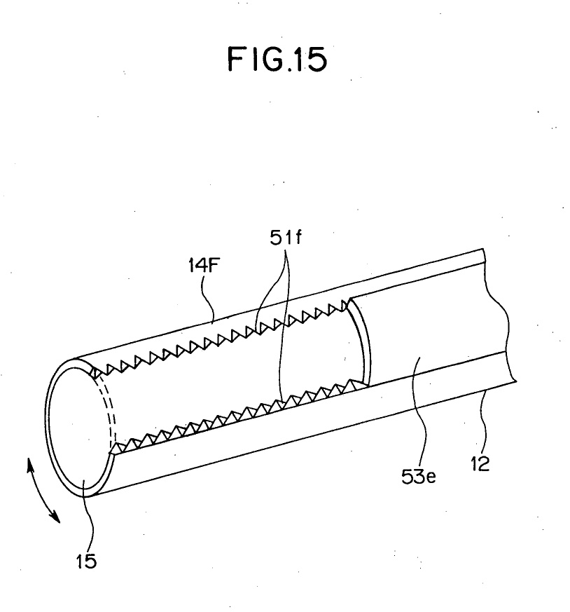

According to a modification of the third embodiment, a treatment portion 14E may be arranged, in which a part of a pipe can advance and return as shown in FIGS. 13 and 14.

That is, referring to FIG. 13, the treatment portion 14E has a notch surface 51e that is formed by notching a part of a hollow pipe. Further, the treatment portion 14E has an advance and return portion 53e that slidably advances and returns on the notch surface 51e.

In the case of the modes 3 to 6 shown in Table 1 or of outputting the torsional vibrations in the manual mode, the advance and return portion 53e is moved back and the notch surface 51e is exposed. In this case, the energy is concentrated on the notch surface 51e and the treatment portion 14E cuts the hard tissue.

Meanwhile, in the mode 1 shown in Table 1, or in the case of outputting only the longitudinal vibrations in the manual mode, in the treatment portion 14E, the linear motor is driving-controlled under the control of the control circuit 35, thereby advancing the advance and return portion 53e. Referring to FIG. 14, the notch surface 51e is hidden and is used as a normal pipe.

Referring to FIG. 15, a treatment portion 14F may have a notch surface 51f that is zigzag-shaped. In this case, the treatment portion 14F easily cuts the harder tissue.

Thus, the ultrasonic treatment apparatus according to the third embodiment obtains the similar advantages as those according to the second embodiment, and the longitudinal vibrations and the torsional vibrations can be switched.

Having described the preferred embodiments of the invention referring to the accompanying drawings, it should be understood that the present invention is not limited to the those precise embodiments and various changes and modifications thereof could be made by one skilled in the art without departing from the spirit or scope of the invention as defined in the appended claims.

1. An ultrasonic treatment apparatus comprising:
an ultrasonic transmitting member having a treatment portion for treating a target portion, the ultrasonic transmitting member transmitting ultrasonic vibrations to the treatment portion;
a transducer which is connected to the ultrasonic transmitting member and includes a first element for vibrating the ultrasonic transmitting member in an axial direction thereof and a second element for vibrating the ultrasonic transmitting member in a torsional direction thereof;
a rotation driving portion which freely rotates the transducer;
a first driving portion which drives the first element in the transducer;
a second driving portion which drives the second element in the transducer; and
a control portion which independently controls the first driving portion, the second driving portion, and the rotation driving portion.

2. An ultrasonic treatment apparatus according to claim 1, wherein the control portion independently controls the first driving portion, the second driving portion, and the rotation driving portion, in accordance with a set vibration mode.

3. An ultrasonic treatment apparatus according to claim 2, wherein the control portion controls an on/off signal and the intensity of a driving signal which is outputted from the first driving portion, the second driving portion, and the rotation driving portion, in accordance with the set vibration mode.

4. An ultrasonic treatment apparatus according to claim 3, wherein the set mode is at least one of a perforation, emulsification and aspiration mode, a perforation mode, a cutting mode, a perforation and cutting mode.

5. An ultrasonic treatment apparatus according to claim 4, wherein the perforation, emulsification and aspiration mode uses the vibrations in the axial direction generated by the first element,
the perforation mode uses the combination of the vibrations in the axial direction generated by the first element and the rotation of the rotation driving portion,
the cutting mode includes a first cutting mode that uses the vibrations in the torsional direction generated by the second element, and a second cutting mode that uses the combination of the vibrations in the torsional direction generated by the second element and the rotation of the rotation driving portion,
the perforation and cutting mode includes a first perforation and cutting mode that uses the combination of the the vibrations in the axial direction generated by the first element and the vibrations in the torsional direction generated by the second element, and a second perforation and cutting mode that uses the combination of the vibrations in the axial direction generated by the first element, the vibrations in the torsional direction generated by the second element, and the rotation of the rotation driving portion.

6. An ultrasonic treatment apparatus according to claim 1, wherein the rotating velocity of the rotation driving portion is higher than the vibration velocity of the vibrations generated by the second element.

7. An ultrasonic treatment apparatus according to claim 1, wherein the ultrasonic transmitting member has a suction channel which is opened to the treatment portion, and through which the tissue is sucked, and
the treatment portion forms a cavitation generating surface which generates the cavitation caused by the torsional vibrations, to the tissue.

8. An ultrasonic treatment apparatus according to claim 1, wherein at least a part of the treatment portion is provided with non-circular-shaped cross section in the direction perpendicular to the longitudinal direction thereof.

9. An ultrasonic treatment apparatus according to claim 7, wherein the treatment portion has a portion which is slidable with respect to the other portion thereof.

10. An ultrasonic treatment apparatus according to claim 9, wherein the slidable portion slidably moves in the axial direction of the ultrasonic transmitting member.

11. An ultrasonic treatment apparatus comprising:
a transducer which generates the ultrasonic vibrations; and
a treatment portion, for treating a target portion, connected to the transducer so that the ultrasonic vibrations generated by the transducer are transmitted, at least a part of the treatment portion being provided with non-circular-shaped cross section in the direction perpendicular to the longitudinal direction thereof.

12. An ultrasonic treatment apparatus according to claim 11, wherein the transducer includes a first element that vibrates the treatment portion in an axial direction thereof and a second element that vibrates the treatment portion in a torsional direction thereof; and further comprises:
a rotation driving portion which freely rotates the transducer;
a first driving portion which drives the first element;
a second driving portion which drives the second element; and
a control portion which independently controls the first driving portion, the second driving portion, and the rotation driving portion.

13. An ultrasonic treatment apparatus according to claim 12, wherein the control portion independently controls the first driving portion, the second driving portion, and the rotation driving portion, in accordance with a set vibration mode.

14. An ultrasonic treatment apparatus according to claim 13, wherein the control portion controls an on/off signal and the intensity of driving signals which are outputted from the first driving portion, the second driving portion, and the rotation driving portion, in accordance with the set vibration mode.

15. An ultrasonic treatment apparatus according to claim 14, wherein the set mode is at least one of a perforation, emulsification and aspiration mode, a perforation mode, an cutting mode, a perforation and cutting mode.

16. An ultrasonic treatment apparatus according to claim 15, wherein the perforation, emulsification and aspiration mode uses the vibrations in the axial direction generated by the first element,
the perforation mode uses the combination of the vibrations in the axial direction generated by the first element and the rotation of the rotation driving portion,
the cutting mode includes a first cutting mode that uses the vibrations in the torsional direction generated by the second element, and a second cutting mode that uses the combination of the vibrations in the torsional direction generated by the second element and the rotation of the rotation driving portion,
the perforation and cutting mode includes a first perforation and cutting mode that uses the combination of the the vibrations in the axial direction generated by the first element and the vibrations in the torsional direction generated by the second element, and a second perforation and cutting mode that uses the combination of the vibrations in the axial direction generated by the first element, the vibrations in the torsional direction generated by the second element, and the rotation of the rotation driving portion.

17. An ultrasonic treatment apparatus according to claim 12, wherein the rotating velocity of the rotation driving portion is higher than the vibration velocity of the vibrations generated by the second element.

18. An ultrasonic treatment apparatus according to claim 12, the treatment portion has an opening of a suction channel, through which the tissue is sucked, and a cavitation generating surface which generates the cavitation caused by the torsional vibrations to the tissue.

19. An ultrasonic treatment apparatus according to claim 18, wherein the treatment portion has a portion which is slidable with respect to the other portion thereof.

20. An ultrasonic treatment apparatus according to claim 19, wherein the slidable portion slidably moves in the axial direction of the treatment portion.

21. An ultrasonic treatment apparatus comprising:
an ultrasonic transmitting member having a treatment portion for treating the tissue at one end thereof, the ultrasonic transmitting member transmitting ultrasonic vibrations to the treatment portion;
a transducer which is connected to the ultrasonic transmitting member and includes a first piezoelectric element for vibrating the ultrasonic transmitting member in an axial direction of the ultrasonic transmitting member and a second piezoelectric element for vibrating the ultrasonic transmitting member in a torsional direction of the ultrasonic transmitting member, the first piezoelectric element and the second piezoelectric element being laminated in an axial direction of the ultrasonic transmitting member;
an electromagnetic motor which freely rotates the entire transducer;
a first driving portion which drives the first element;
a second driving portion which drives the second element; and
a control portion which independently controls the power which is supplied to the first piezoelectric element, the second piezoelectric element, and the electromagnetic motor.

22. An ultrasonic treatment apparatus according to claim 1, wherein the treatment portion has at least one edged portion.
